(19) United States
(12) Patent Application Publication
(10) Pub. No.: US 2025/0152063 A1
(43) Pub. Date: May 15, 2025

(54) Sensor Unit and Method for Detecting Brain-Wave-Induced Magnetic Fields

(71) Applicant: Robert Bosch GmbH, Stuttgart (DE)

(72) Inventors: Felix Michael Stuerner, Ulm (DE); Tino Fuchs, Tuebingen (DE); Riccardo Cipolletti, Geislingen An Der Steige (DE); Andre Rudi Kretschmann, Moessingen (DE); Hans-Joachim Bieg, Weil Im Schoenbuch (DE); Robert Roelver, Calw-Stammheim (DE)

(21) Appl. No.: 18/839,315

(22) PCT Filed: Dec. 30, 2022
(86) PCT No.: PCT/EP2022/088088

§ 371 Date: Aug. 16, 2024

(30) Foreign Application Priority Data
Feb. 18, 2022 (DE) 10 2022 201 697.1

(51) Int. Cl. A61B 5/245; A61B 5/00; G01R 33/022

(52) U.S. Cl. CPC A61B 5/245; A61B 5/6803; G01R 33/022; A61B 2562/0219; A61B 2562/0223

(57) ABSTRACT

A sensor unit for detecting brain current-induced magnetic fields in an unshielded environment has a plurality of gradiometer units configured for arrangement around a head of a user. Each gradiometer unit has two magnetometers which are arranged at a fixed distance from each other. Each magnetometer has a sensor medium and is configured to detect a magnetic field strength at a measurement location by reading a spin resonance in the sensor medium depending on the magnetic field strength. The sensor unit further includes at least one excitation light source for radiating light into the sensor media of the magnetometer. The sensor unit further incudes at least one signal processing unit for determining a magnetic field gradient at a gradiometer unit as a difference of the output signals of the two magnetometers of the gradiometer unit and for detecting a time course of the magnetic field gradient.

The present invention relates to a sensor unit and method for detecting brain current-induced magnetic fields as well as a computing unit and a computer program for carrying out the same.

### BACKGROUND OF THE INVENTION

It is necessary to recognize mental or physical states of a user in various areas. On the one hand, this includes medical applications in order to detect illnesses or critical states in a timely manner; but it is also of interest, for example, to automatically detect whether a user is fit to drive in a vehicle or whether the ability to drive is restricted due to fatigue or a medical emergency.

Various methods are known for monitoring states such as fatigue. For example, eye movements and blink frequencies may be monitored by camera images, and from certain patterns, the ability of the user to drive may then be inferred. However, this must have a continuous free field of view, which can be prevented, for example by poor light conditions, head movements or glasses.

In addition, measurement methods such as the EEG (electroencephalogram), where potential changes are measured throughout the scalp, provide reliable indications of a user's mental state, including for the detection of fatigue or emergencies. However, electrodes must be correctly applied to the head and provided with a contact gel for a good measurement result, so that an EEG is not practical as a measurement method in daily life, such as for driver monitoring.

### DISCLOSURE OF THE INVENTION

According to the invention, a sensor unit and method for detecting brain current-induced magnetic fields in an unshielded environment, as well as a computing unit and computer program for performing the method with the features of the independent claims, are proposed. Advantageous embodiments are the subject of the dependent claims and the following description.

The invention creates a method for measuring brain currents and associated mental or physical states of a user that is suitable for everyday use and as contactless as possible.

In particular, a sensor unit comprising a plurality of gradiometer units configured to be arranged around a head of a user is proposed. Each gradiometer unit includes two magnetometers which are arranged at a fixed distance from each other. Each magnetometer has a sensor medium and is configured to detect a magnetic field strength at a measurement location by reading a spin resonance in the sensor medium dependent on the magnetic field strength. The sensor unit further comprises at least one excitation light source for radiating light into the sensor media of the magnetometers and at least one signal processing unit configured to determine a magnetic field gradient at a gradiometer unit as a difference in the output signals of the two magnetometers of the gradiometer unit and to detect a time course of the magnetic field gradient. By using sensitive magnetometers based on spin resonance effects as well as the use of a gradiometer configuration, it is possible to effectively detect the desired signals caused by brain currents even in existing background fields, such as the earth's magnetic field or artificially generated magnetic fields. This eliminates the need for intricate magnetic field shielding and complicated sensor handling, making it possible to use this sensor unit to measure brain activities in everyday environments.

According to a possible embodiment, at least one of the magnetometers may comprise a nitrogen vacancy center magnetometer, wherein the sensor medium comprises a diamond crystal or a portion of a diamond crystal with nitrogen vacancy centers, and wherein the sensor unit further comprises at least one microwave source for generating a resonant field in the sensor medium and at least one photodetector for detecting resonance-dependent sensor light from the cell. Nitrogen vacancy (NV) center magnetometers are highly sensitive and thus well-suited for detecting the weak brain current magnetic fields. By using single diamonds or a common diamond for multiple sensor heads, easy-to-use sensors can be implemented that can be integrated into many devices. In particular, small light sources and photodetectors can also be integrated directly with the diamonds—e.g., in the form of thin diamond plates—into compact sensor heads. The magnetometers based on nitrogen failure centers also allow vector measurements of the magnetic fields and provide high dynamics.

In particular, with a gradiometer unit formed from two nitrogen vacancy center magnetometers, the two magnetometers of the at least one gradiometer unit may be associated with the same excitation light source and the same microwave source, such that reliable suppression of all noise sources and background signals occurs approximately equally on both magnetometers of a gradiometer unit.

Additionally or alternatively, at least one of the magnetometers may comprise a vapor cell magnetometer, wherein the sensor medium comprises an atomic spin-polarizable vapor in a cell, wherein the atomic vapor comprises an alkali metal, a noble gas or an alkali-metal azide, for example. Such cells are easily produced on a small scale and also provide very high sensitivity.

Such a sensor unit may comprise at least one holding device in which multiple gradiometers units are mounted, such that they are arranged in operation at different locations near the head surface of a user. The distance between the two magnetometers of a gradiometer unit is to be in the millimeter to centimeter range, for example between about 2 and 30 millimeters, preferably between 5 and 20 millimeters. With these approximate distances, it is possible to assume the background field is uniform and effectively eliminate it, while the brain magnetic field signals of interest decrease with the square of the distance from the source and are thus significantly lower at one of the two magnetometers. The gradiometers may also be located directly on the head surface or a few millimeters to centimeters from the head, depending on the strength of the signals and sensitivity of the sensor structure.

For example, the holding device, in which a sensor unit or the gradiometer sensors can be accommodated, may comprise a head rest, a headgear, a headband, a cushion, or a head part of a recliner. However, any other holding devices can also be used to bring the gradiometer units sufficiently close to the head of a user. Optionally, in particular, holder devices can also be used, in which the head of the user can be held in the holder device in such a way that the head remains freely movable relative to the holder device. For example, headgear or straps are attached to the head in a removeable manner, whereas in the case of a head rest or head part of a recliner, the respective elements may be shaped such that the head can be accommodated in them, but is not fixed and does not need to rest directly against the holding device. Thus, the sensor unit according to the invention can also be used in everyday environments without restricting the user, such as in hospital beds, during emergency transportation, for monitoring the states of an automobile driver, train operator or pilot, or in any other areas.

Additionally, in some embodiments, the sensor unit may further comprise a position reference unit wearable on the head of a user and configured to determine a relative position or relative movement between the head of a user and the plurality of gradiometer units. Such a position reference is particularly useful in embodiments in which the position of the gradiometer units on the head can shift or in which free movement of the head is provided and thus constant changes in the relative position occur. The reference signals may then be used to correct the obtained magnetic field signals or to evaluate the reference signals with the magnetic field signals in combination.

For example, at least one gyroscope or at least one element may be used as a position reference unit to generate a reference magnetic field (e.g., by permanent magnets or a coil) that can be detected by magnetometers of the sensor unit. These position reference units may be worn in suitable elements on the head of a user, such as in a spectacle frame, earpiece, headband, or similar devices.

Furthermore, a method for detecting brain current-induced magnetic fields using such a sensor unit is proposed, which comprises detecting magnetic field strengths by the magnetometers of the sensor unit in the head region of a user; determining, for each gradiometer unit, a magnetic field gradient by forming a respective difference signal from the output signals of the associated magnetometers; detecting a time course of the magnetic field gradients for each gradiometer unit; and checking the time course of one or more magnetic field gradients for the occurrence of predetermined patterns. The check may be performed in a classic manner by comparing pattern parameters and evaluating curve parameters of the time course; additionally or alternatively, machine learning methods may also be employed to check the detected magnetic field signals for known, unknown, or undesired patterns.

In addition, a position reference signal may be detected for determining a relative position between the gradiometer units and the head of a user, and may be used to together with the magnetic field gradients to determine a position-corrected curve of the magnetic field gradients. This evaluation of the position reference signals may optionally be performed using artificial learning units.

The signal for forming a position reference may comprise, for example, an additional brain current-induced magnetic field signal, which is independent of a user's mental state, or also a gyroscope signal from one or more gyroscopes attached to the head of a user. Another option is a magnetic field signal generated by a position reference unit attached to the head of a user. All of these methods may also be suitably combined with one another.

Moreover, the method may comprise detecting, based on checking the time history, an undesirable state of the user, and initiating responses based on the detection. The initiated reactions may be controlled in other devices and systems or may be performed by additional elements of the sensor unit (e.g., displays, signal generators) and are otherwise dependent on the area of application of the sensor unit. The undesired state may include, for example, fatigue, stress, falling asleep, a neural disease such as Parkinson's disease, a stroke, epilepsy, or any other state that results in a measurable change in a user's brain activity.

In programming terms, a computing unit according to the invention, e.g., a control device of a vehicle or a control unit of a patient monitoring system, is in particular configured to perform a method according to the invention.

The implementation of a method according to the invention in the form of a computer program or computer program product comprising program code for performing all of the method steps is also advantageous because this results in particularly low costs, especially if an executing control device is still used for other tasks and is therefore provided in any event. Lastly, a machine-readable storage medium is provided, on which a computer program as described above is stored. Suitable storage media or data carriers for providing the computer program are, in particular, magnetic, optical, and electric storage media, such as hard disks, flash memory, EEPROMs, DVDs, and others. Downloading a program via computer networks (internet, intranet, etc.) is also possible. Such a download can take place in a wired, or cabled, or wireless manner (e.g., via a WLAN, a 3G, 4G, 5G, or 6G connection, etc.).

Further advantages and embodiments of the invention will emerge from the description and the accompanying drawings.

The invention is thoroughly illustrated schematically in the drawings on the basis of exemplary embodiments and is described hereinafter with reference to the drawings.

### BRIEF DESCRIPTION OF THE DRAWINGS

FIG. 1 schematically shows a possible construction of a sensor unit with a gradiometer construction;

FIG. 2 schematically shows a head rest with a plurality of gradiometers;

FIGS. 3a and 3b show cross-sections through exemplary holding devices to arrange sensor units around a head of a user; and

FIG. 4 schematically shows the signal processing of the detected gradiometer signals according to one exemplary embodiment.

### EMBODIMENTS OF THE INVENTION

From the brain currents of a human user, which can be measured, for example via potential changes on the scalp, there are indications of different mental, physical or health states of the user. However, the electrical activity in the brain neurons also induces a weak magnetic field, which can be detected and evaluated by magnetic field sensors. From the time curve of the induced magnetic fields, conclusions can then also be drawn about the brain current patterns and thus on different states of a user.

As the magnetic field strengths to be measured are very small, particularly quantum-based or optically pumped magnetometers that allow for highly sensitive measurements are suitable as sensors for such a setup. In the following embodiments, only two types of sensors are described as possible magnetometers for the proposed sensor unit, namely vapor cell magnetometers and diamond NV magnetometers. However, it is also conceivable to integrate other magnetometers with comparable properties into the sensor unit.

The magnetometers described herein, as an example, utilize optically pumped and/or optically detected magnetic resonance (ODMR). It is thereby exploited that under the influence of an external magnetic field, the energy levels of certain spin states of unpaired electrons are broken down (Zeeman effect). The splitting of the energy levels results in changed transitions during relaxation from excited states, which can then be measured, for example, by optical excitation and frequency-dependent detection of the resulting fluorescence radiation or by observation of optical properties such as the absorption of light. From the measured optical parameters, the magnetic field strength may then in turn be inferred.

Diamond NV magnetometers are based on reading out magnetic resonances from special defect centers in diamond, in particular nitrogen vacancies (NV), which occur as impurities in the carbon lattice of diamond and can also be introduced in a targeted manner. Details can be found in DE 10 2018 202 238 A1, for example.

Because the nitrogen vacancy center in the single-crystal diamond has four possible ways to position itself in the crystal lattice, the presence of a directed magnetic field causes the nitrogen vacancy centers present in the crystal to react differently to the external magnetic field depending on their position in the crystal. As a result, four pairs of fluorescence minima may appear in the spectrum, from the shape and position of which both the magnetic field strength as an amount and the direction of the external magnetic field are clearly determinable.

The NV center magnetometer provides a variety of advantages for the present application. In addition to the very highly sensitivity already mentioned, a high measurement range (>1 Tesla) can also be covered. The underlying Zeeman effect is linearly dependent on the magnetic field present and also does not show degradation, since the measurement is based on quantum mechanical states. In addition, an NV center magnetometer provides the possibility of determining external magnetic fields vectorially based on the different orientations present in the diamond lattice.

There are also alternative ways to electrically read the magnetic spin resonance in diamond. Charge carriers are detected that were lifted into the diamond conduction band by two-photon ionization of the NV centers. If such a method is used to read out the resonance effects, parts for detecting the fluorescent light are not required and are replaced with suitable photo-current detectors on the diamond. However, apart from this, the method of magnetic field measurement may be transferred accordingly and may be applied in all embodiments with NV center magnetometers.

Vapor cell magnetometers are also based on the reading of spin resonances. A cell, e.g., a closed cavity in a silicon wafer, is filled with gaseous atoms and optional additional buffer gases, e.g., vaporized alkali metals such as potassium, rubidium or cesium, which also have unpaired electrons. Depending on the embodiment, mixtures of alkali vapor and noble gases may also be used. In other cases, pure noble gases are used.

Also for vapor cell magnetometers, spin polarization via optical pumps with an excitation light source is achieved. Optical and subsequently optical properties of the atoms in the cell are then read via the resonance effects, e.g., with an additional read-out light source, by means of which the absorption of the read-out light by the vapor cell is measured. Different magnetic field-dependent state splitting may be observed.

In order to be usable in an everyday environment, magnetic fields that do not originate from brain activity should be eliminated as far as possible from the measurement. In the automotive sector in particular, comparatively high and barely shielded magnetic fields occur in the background, which lie in the range of about 10⁻⁶ to 10⁻⁹ Tesla (nanotesla), and the earth magnetic field is in the range of 10⁻⁵ Tesla (a few microteslas). In contrast, the biomagnetic fields of interest here are in the range of 10⁻² Tesla (picoteslas) or even lower.

The elimination of the background magnetic fields may be achieved by a gradiometer arrangement for the magnetic field measurement, according to exemplary embodiments. Gradiometers are generally referred to as sensor units that are capable of detecting not only the field strength, but also the gradient of the field. At least two individual magnetometers may be used for this purpose, which are arranged at spatially different locations. As an example, a sensor unit that uses two NV center magnetometers in a gradiometer arrangement is described below.

A diamond with nitrogen cavities may first be present for each magnetometer. The optical excitation of the NV centers is achieved by a suitable light source, such as a pump laser. Here, an Nd:YAG laser in the green range at a frequency of 532 nm is suitable to excite the respective transitions. The light of the pump laser can be radiated into the diamonds via suitable optical elements such as mirrors, beam splitters, lenses and, optionally, via fiber optic elements. Here, a single light source for both diamonds is used, from which the excitation light is directed via an optical fiber into the respective diamond. In addition, the excitation light may be continuously irradiated or pulsed by the laser such that, for example, time windows for interference-free fluorescence light measurement are kept free.

The distance d between the diamonds corresponds to the distance of the locations where magnetic field measurements are taken simultaneously. As long as the distance of the measurement locations is relatively small, it can be assumed that the strength of the additional background magnetic field Bₑₙᵥ is approximately equal at both locations. In contrast, the weak magnetic field induced by brain currents will decrease significantly with increasing distance from the head or more generally from the source of the magnetic field. By arranging two sensors at different distances from the measurement range, e.g., from the cranial surface, the background field can be eliminated by forming a difference between the detected sensor values and the magnetic field of brain activity or its gradient can be extracted. As the magnetic field weakens with the square of the distance, the greatest magnetic field change is detected by the sensor diamond near the source. For this purpose, for example, two magnetometers can be arranged one on top of the other in an axial gradiometer configuration, i.e., substantially perpendicular to the cranial surface.

Thus, for an axial gradiometer with a first and a second magnetic field sensor:

B₁=B_{brain1+Benv},

B₂=B_{brain2+Benv},

B_{grad}=B₁−B₂=_{Bbrain1-Bbrain2}≈B_{brain1}
wherein B₁ is the measured field strength at the first sensor located closer to the magnetic field source of interest (in this case the head), wherein B_{brain1} is the portion of the magnetic field strength at the location of the first sensor that is induced by the brain currents, wherein B₂ is the measured field strength at the second, more distant sensor, wherein B_{brain2} is the portion of the magnetic field strength at the location of the second sensor that is induced by the brain currents, and wherein B_{grad} is the resulting gradient of the magnetic field strength.

As the magnetic field B_{brain2} induced by brain currents at the second sensor, which is further away from the source, is many times smaller than at the first sensor, the gradient B_{grad} of the total magnetic field at this location corresponds approximately to the magnetic field B_{brain1} induced by brain currents at the first, closer sensor head, while approximately uniform background fields Ben, are eliminated.

The distance d of the sensor heads within a gradiometer unit, i.e., in this case the distance of the two sensor diamonds, may be in the range of mm to a few cm, for example between 0.1 cm and 2 cm, preferably below 1 cm. Good results were achieved for values in these orders of magnitude for the measurement of biomagnetic fields. However, as long as sufficient independence of the signals from the background fields is achieved, larger or smaller distances or other gradiometer configurations may also be used, such as sensors arranged side-by-side parallel to the head surface. If the distance between the individual sensor heads is too large, the background field may no longer be identical at both locations; especially for local background fields (electrical leads, etc.); if the distance between the sensor heads is too small, the difference between the field strengths from the brain current magnetic field between the two locations may be too small. The choice of the appropriate distance between the two sensors of a gradiometer unit is thus preferably also dependent on the type and orientation of the magnetic field source of interest (e.g., a certain brain area), the expected field strength and the distance of the entire gradiometer unit to the magnetic field source, i.e., in this case, also the inevitable distance to the active brain neurons inside the head, which induce the magnetic field.

In addition to an axial gradiometer configuration as described above, in which the sensors are arranged practically along the direction of the measurement signal perpendicular to the head surface, other configurations are also conceivable. In particular, it is also useful to use several measurement axes, or specifically three measurement axes. In particular, an NV center sensor that enables vector magnetometry is well-suited for this purpose. In these cases, the measuring directions perpendicular to the head surface in particular can then be used for corrections. Generally, gradiometric arrangements may ideally compensate in all spatial directions, but in particular in the direction of measurement.

Also, the arrangement of multiple sensors in a sensor array having one or more rows may be used to achieve motion correction and detailed reading of different brain regions. Here too, for efficient suppression of the background, the sensors should be mounted closely to one another, i.e., again in the mm range.

The distance of a sensor unit from the head may also be in the mm to cm range. When affixed directly to the head of a user using hoods or similar holders, a very small distance from the head and thus a minimum distance from the magnetic field sources is achieved. In an arrangement of the sensors in a holder that is close to the head, the distances to the head may be from a few mm to a few cm in order to achieve sufficiently large and detectable magnetic field strengths at the measurement location. The possible distance and arrangement is also dependent on the depth and orientation of the expected brain activity regions.

Also, the described vapor cell magnetometers may be used in such gradiometer arrangements.

In addition, a microwave source may be present in the sensor unit capable of generating an electromagnetic field across a bandwidth sufficiently covering the desired resonance frequency in the range of the NV centers in the diamonds. Preferably, the same microwave source may be used for both sensors or diamonds, the field of which is then directed to the diamonds via a high frequency connector. In addition, a microwave resonator structure (not shown) may also be present on each diamond to homogeneously distribute the generated microwave field across the volume of the sensor diamond.

The resulting fluorescent light may in turn be directed via the fiber optics and may be detected by a photodetector that is sensitive in the fluorescence wavelength range; alternatively, one or more photodetectors could also be arranged directly on the sensor diamonds that receive the fluorescent light. Here too, suitable reflective or semi-transparent mirrors, filters, beam splitters, lenses for beam shaping, coupler lenses for fiber optics or for diamond crystals, and other optical elements may be utilized along the optical path of the fluorescent light. The signal of the photodetector may then be further processed by various elements, such as a pre-amplifier or a computing unit for signal evaluation.

The elements of the sensor unit are shown only schematically in the figure; it is to be understood that, for example, the microwave source is configured and arranged to generate the RF field in the sensor diamonds. Other elements such as microwave resonators or housing components are not expressly shown here.

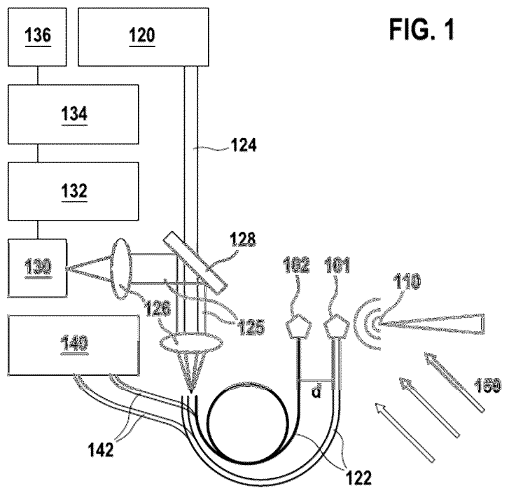

Instead of two sensor diamonds, as shown in FIG. 1, only a single diamond with NV centers could be used that has a certain spatial extent at least along one direction to allow for a gradiometer configuration. To this end, the diamond is excited in at least two locations and the resulting fluorescent light is read out by at least two suitably arranged photodetectors. For this purpose, for example, a diamond waveguide with NV centers can also be used so that the excitation light can propagate along the gradiometer axis in the waveguide and reach both readout locations. In the other direction, no large extent of the crystal is required, such that diamond plates, for example, are conceivable as sensor heads.

If the same microwave source and the same light source are used for both sensor diamonds and diamond portions, noise components from these sources (noise from the excitation light or frequency and amplitude noise of the microwave source) also occur at both sensor heads, so that these simultaneously occurring noise components are automatically eliminated by the gradiometer arrangement. This reduces the requirements for the noise characteristics of these components. For a microwave source, for example, an inexpensive standard oscillator with higher phase and amplitude noise may then be used instead of a high-accuracy, expensive and temperature-controlled quartz oscillator.

For example, to further suppress sensor noise from the gradiometer, a modulation method may be used in which the microwave frequency or the amplitude of the microwaves is modulated. Here, modulation of the microwave frequency at a particular modulation depth and modulation frequency generates a modulation of fluorescence. By demodulating the detected fluorescence signal, a signal that is linearly dependent on the magnetic field is then obtained in the magnetic resonance frequency range. Alternatively or additionally, the light intensity of the excitation light may also be modulated.

Particularly interesting is also the option of determining the location of the magnetic field source, i.e., enabling vector magnetometry. The field gradient is to be determined in three axes. The direction in which the magnetic field gradient is maximum indicates the location of the magnetic field source. For this purpose, for example, permanent magnets or coils can be used to generate an additional bias magnetic field that is identical at both measuring points of the gradiometer (i.e., in the area of the sensor heads or sensor diamonds). In one possible embodiment, a single cylindrical coil may be used for this purpose, within which the sensitive diamond sensors are located. Here too, the use of a single coil and power source ensures that the same noise components are expected at both sensors, which in turn are eliminated by the gradient formation.

In addition or as an alternative to a gradiometer configuration, a lock-in detection may be used for a magnetometer to eliminate the undesirable background fields from the measurement. For this purpose, for example, the excitation light or microwave source may be modulated and a lock-in amplifier 134 may be provided in the area of signal processing of the sensor unit 100, which acts substantially as a narrow band pass for the resonance signal of interest.

As a further embodiment, gradiometer units may also be formed from two vapor cell magnetometers. While these vapor cell magnetometers often require strong magnetic shielding, as they are typically only operated with small field strengths close to the near-zero field, measurements can also be made in normal environment using vapor cells with a gradiometer configuration or with suitable lock-in detection. Combinations of vapor cells and NV center magnetometers are also possible within one gradiometer unit.

In order to read magnetic fields induced by brain currents, a plurality of individual magnetometers and/or a plurality of gradiometer units, each consisting of two magnetometers can be arranged at different locations around the head of a user to form a sensor unit. According to their specific spatial arrangement, the potentials generated by individual neurons and the induced magnetic fields are added up, so that magnetic field changes distributed over the entire head can be measured. The sensor unit may comprise a holding device in which a plurality of magnetometers or gradiometers is arranged. The associated further elements of the sensor unit, such as the signal processing modules, light sources, etc., can either also be integrated in the holding device or can be accommodated separately, e.g., in a separate housing. By way of example, compact gradiometer units can be formed with two sensors, in each of which a diamond, optical filters and photodetectors as well as a microwave resonator are integrated. It is also possible to arrange a small light source (e.g., a laser diode) as well as a photodetector directly on the diamond and thus form a compact sensor head. The electronics for control and signal processing can then in turn be arranged outside the actual sensor heads. Each magnetometer of a sensor head can be formed as a separate unit, but a compact gradiometer unit could also be formed within a single housing, which can then be easily installed in other devices.

Generally, for example, a type of headgear or a holder device supported on the head for the entire sensor unit or for parts of the sensor unit, e.g., for the multiple magnetometers or gradiometer units, can be used for this purpose. For example, if a user wears a helmet, such as a motorcyclist or pilot, one or more sensor units, gradiometer units, or magnetometer sensor heads may be directly integrated into that helmet. Also, temples, earpieces, hearing aids, and other head-worn devices may be combined with the sensor units described herein and accommodate the sensor heads in a suitable location. Likewise, holding devices in the form of a continuous or mesh-like hood, or, e.g., similar to a hairband or headband, can be used, in which a plurality of sensor heads are accommodated and which can be placed on the head.

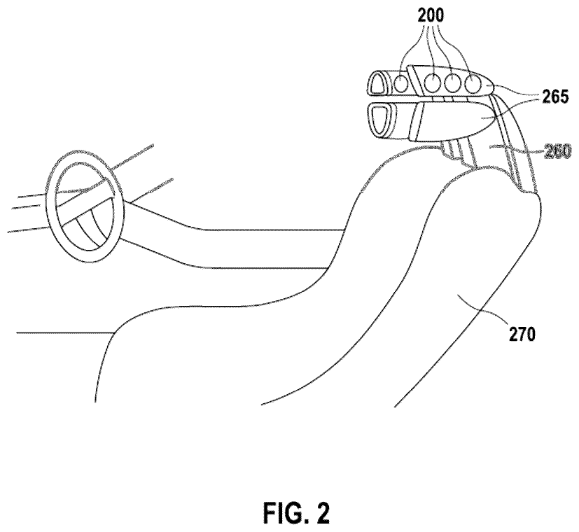

In other cases, however, it is more comfortable for the sensor unit to be arranged near the head without being directly attached thereto. For example, to monitor a state of a driver in a vehicle, sensor units or parts thereof may be integrated into a head rest 260 of a seat element 270, as exemplified in FIG. 2. A head rest can be specifically configured such that extenders 265 laterally extend at least partially around the head and/or above the head as a holding device for the gradiometer units, and a plurality of gradiometers 200 or magnetometers is arranged at various locations in these side portions 265. It is understood that the shapes of the head rest or side portions 265 of the head rest preferably follow approximately the head shape in order to be able to bring the sensors 200 as close as possible to the desired measurement ranges. If it is sufficient for the desired monitoring to detect magnetic field signals primarily at the back of the head, a standard head rest with correspondingly embedded sensor heads can also be used. In all variants, magnetometers and/or gradiometers may also be arranged at different heights.

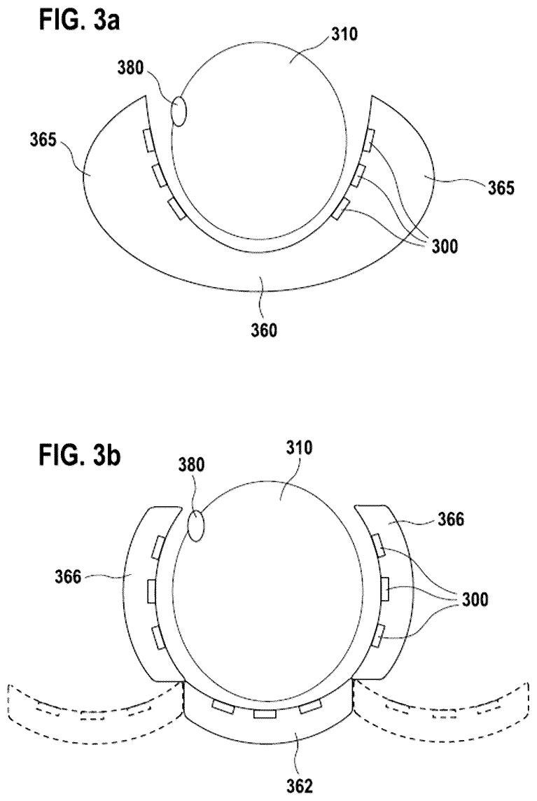

FIGS. 3a and 3b show similar holding devices 360 such as head rests with a plurality of gradiometers 300 or magnetometers in a cross-section. In FIG. 3a, a conventional head rest 360 is extended on both sides by lateral extenders 365, in which several gradiometer units 300 are mounted. The individual gradiometers 300 may be arranged adjacent to each other or offset from each other vertically around the head circumference. In the present example, several gradiometer units 300 are provided symmetrically on both sides. However, the location and the distance of the gradiometers may also be chosen differently depending on the brain regions to be monitored and the design of the holding device as well as the sensor unit.

It is to be understood that such head rest elements may also be additionally adapted to increase functionality and comfort. For example, as shown in FIG. 3b, manually or automatically pivotable side elements 366 may be provided as holding devices, in which the sensor units 300 are arranged. These can be folded down to an open position via hinges or other connections (shown as dashed in the figure) or moved directly to the head of the user 310 (continuous line). Furthermore, it could also be possible to move the holding devices 366 and/or parts thereof in any direction in order to enable the sensor unit to be used for different body sizes, for example. In this way, a driver can first position himself easily in the seat, and then the movable side elements 366 are moved closer to the head 310. This ensures that the sensor heads of the individual magnetometers or gradiometers may be placed as close to the head as possible and correctly. Additionally, a rear portion 362 of the head rest, which is not movable, may also include gradiometer units 300.

Further parts of the sensor unit, such as a power supply and means for signal processing, can then also be integrated in the holding devices or optionally also in the remaining parts of the head rest or, e.g., in the seat. The detected sensor data of the sensor unit may additionally also be stored locally or may be transmitted to other modules or systems, e.g., via a wireless communication interface such as WLAN or Bluetooth. It is thus also possible that all sensor data is transmitted to a processing unit in a wired or wireless manner prior to an evaluation and that signal processing only takes place there. A dedicated computing unit may be used as the processing unit, but it is also possible that a computing unit or controller may perform these tasks, which is already set up for other purposes, such as a central control unit in a vehicle. Additionally or alternatively, it is possible that the processed signals and results, e.g., the detected user states or alarm signals, are subsequently also transmitted to further units.

Depending on the embodiment, the gradiometer units can each be configured separately, i.e., comprise two sensor heads with the associated light sources, detectors, optical elements, signal processing units, etc. and together form a complete sensor unit. In other cases, a sensor unit may comprise a plurality of gradiometers and at least parts of the components used may also be used for a plurality of gradiometers in parallel. In particular, for example, a common evaluation unit 136 may be provided for multiple or all of the gradiometer units used in an application. It is also conceivable to use light sources, magnetic field sources or microwave sources together for several gradiometers, as long as this is structurally possible.

A further area of application of the sensor units according to the invention also includes monitoring the state of a user for medical reasons. Here, for example, it would be possible to attach sensor units in a cushion element or on a patient bed. Again, the individual magnetometers or gradiometers 100, 200, 300 are positioned at different locations so that, when in use, they cover different areas of a user's head.

For example, a pre-formed cushion element made of a suitable material, such as a solid foam, could be used, in which a recess is provided for the head that allows useful positioning. At various locations within the recess, the sensor heads of the magnetometer or gradiometer units 300 are embedded so that, when used, they come at least partially around the head of the user, similar to the head rest described in connection with FIG. 3a.

As a further option, a modified variant of the described vehicle head rests described in FIG. 2 or 3 could also be provided on a bed, a hospital bed or chair, such that the head is located in suitably shaped holding devices, such as brackets or shell elements, which are attached to the bed or chair and which can optionally also be pivoted or moved, as in FIG. 3b, in order to ensure correct positioning and convenient access. Thus, for example, brain functions or vital functions of a patient can be continuously monitored without the need for sensors to be fitted by specialist personnel, as is the case with EEG, and without overly restricting the patient's normal movement.

In order to enable an exact evaluation of the measured magnetic fields, it is useful to know the position of the head in relation to the individual gradiometers or magnetometers as precisely as possible. If the magnetometers are not directly attached to the head of a user, but are fixed, this relative position may change, as it cannot be expected that the user will keep his/her head constantly in one position for the intended applications. Therefore, additional modules or method steps may optionally be utilized in all embodiments to determine the relative head position in relation to the measurement units.

For this purpose, for example, one or more gyroscopes attached to the head 310 of the user may be utilized as the position reference module 380. Special temples, attachments for existing glasses, earpieces, headbands or other elements that allow a defined positioning on the head are suitable for this purpose. In operation, the user may therefore wear at least one of the gyroscope modules at a defined location on the head, while the brain current magnetic fields are measured as already described. The head movements can then be continuously tracked in three dimensions via the gyroscope and the resulting displacements of the measurement locations for the magnetometers in relation to the head can be determined using suitable models. These changes can then be taken into account when evaluating the magnetometer or gradiometer signals.

All types of gyroscopes are generally considered for this additional position reference module. Particularly suitable are, for example, the widely used MEMS rotation rate sensors (microelectromechanical system) that are small and sufficiently sensitive. These sensors are typically constructed as a complete sensor system, similar to an integrated circuit, and may include sensors for one or more axes.

However, other gyroscopes may also be used for relative positioning in a position reference module. In a similar way as described for the magnetometer application, vapor cells and NV centers in diamond can also be used for spin-based optical measurement of rotation rates and form highly sensitive gyroscopes. In these cases, the spin Larmor precession ωₗₐᵣₘₒᵣ is directly read out. An external rotation of the gyroscope represents an additional rotation, which can be determined by reading out the rotation frequency ωₘₑₛₛ,

ωₘₑₛₛ=ωₗₐᵣₘₒᵣ±ωᵣₒₜ

For example, DE 10 2019 219 061 A1 discloses a possibility for measuring three directions of rotation by means of an NMR gyroscope.

As the relationship between the gyroscope signals on the head and the magnetic field measurements is not trivial, artificial learning units may also be used for the combined evaluation of the gyroscope signals and brain magnetic field readings, which are trained, for example, on the combination of the two signals in predefined head positions. A corrected magnetic field measurement can then be output as a result, so that comparable signals are available in each head position, which can then be evaluated accordingly. Alternatively, the evaluation of known brain magnetic field patterns can also be applied directly to the combination of the two signals.

Another way to take into account the relative head position compared to fixed magnetometers is to generate a reference magnetic field. For this purpose, for example, weak coils or permanent magnets similar to the gyroscopes described may be attached to suitable elements worn on the head of a user as the position reference module 380. The measured static reference magnetic field and its variation in head movement may then provide information about the relative position of the head 310 relative to the sensor unit and the individual gradiometer units 300. For example, in the case of expected signals of different frequencies, lock-in detection can also be used to separate the signals from each other and evaluate them correctly.

Moreover, the determination of the head position can be made by measuring a further brain signal, which is not influenced by the user state (e.g., fatigue), i.e., preferably a signal that only changes with the head position relative to the sensor. Such a signal may then be used as a reference signal for the position of the head relative to the sensor unit. Signals of different frequencies can in turn be differentiated by suitable signal processing, such as filters or lock-in detection.

The position correction measures described herein for the magnetic field measurements are each individually applicable to one another or in any combination.

If the gyroscope attached to the user is used as a position reference module, its signals may also be used for separate state evaluation or for other purposes. For example, certain motion patterns may be detected and evaluated that may indicate sleepiness or emergencies, e.g., lack of motion over a predetermined period of time. The results of the motion monitoring may then be evaluated individually or in combination with the results of the magnetic field measurements. Of course, a gyroscope may also be provided only for this purpose.

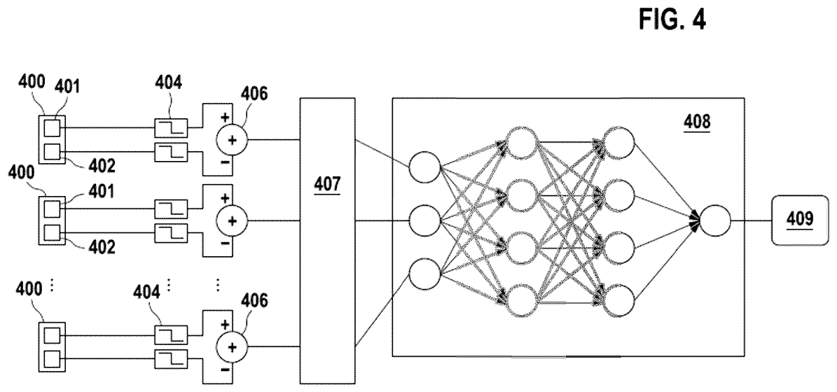

In all possible embodiments, an evaluation unit can connect to the actual magnetometers and the associated detectors, which in particular enables an electronic or computer-aided evaluation of the detected signals. Signal processing is shown schematically in FIG. 4. For each gradiometer 400, the raw signals of the individual magnetometers 401, 402 can optionally first be filtered via a low pass 404, so that all noise components with frequencies significantly above the brain signal frequencies are eliminated. Subsequently, the difference 406 of the individual sensor signals may be formed for each gradiometer 400 according to a gradiometer arrangement, e.g., in a logarithmic differential amplifier. The differential signals may be further processed in an appropriate manner, e.g., by pre-amplifiers, filters, or other signal processing units 407, which may be provided for each gradiometer individually or for multiple gradiometers in combination. Subsequently, the time course of the magnetic field signals may be evaluated in a pattern detection unit 408, e.g., with respect to certain known or unknown patterns or certain parameter characteristics. To this end, in this example, a multi-layer neural network is shown, but any other pattern detection unit can be utilized with or without machine learning portions. This is followed by the evaluation unit 409, which controls certain reactions due to the detected patterns. The pattern detection, the evaluation and classification of the detected patterns as well as the selection of the associated reactions for a detected pattern can also overlap, so that both functions can be performed by a single unit or be divided differently.

For the evaluation of the detected brain-magnetic field signals, classic methods such as different pattern detection methods, simple limit values, temporal curves, tolerance ranges, slopes, frequency evaluations and others can be used. The respective reference values may be stored in the system and associated with certain states or diseases, such as fatigue, stress, but also neural diseases such as Parkinson's, epilepsy or, e.g., strokes.

Alternatively or additionally, artificial/machine learning may also be employed at this point, e.g., in the form of a neural network 408, in order to evaluate and appropriately classify the measured signals. For example, a neural network may also be trained with laboratory-generated data, which were optionally also evaluated by further measurement methods such as EEG as a reference. Based on this data, an artificial learning unit can, in turn, detect fatigue or stress, for example.

Since there is a certain amount of variation in the representative brain current patterns between different users, current measurement data of a user can be stored and included in the further evaluation, especially when using an artificially learning evaluation system. For this purpose, data from the ongoing application operation of a sensor unit can be used, for example, as user-specific training data for a neural network. Even with a classic pattern evaluation, current data can still be included and used for the future evaluation of the measured magnetic field patterns, e.g., by changing certain specified limit values or parameters.

In addition, however, it is also possible for the system to evaluate any deviation of the detected signal patterns from known reference states, even if no associated health state of a user can be associated therewith. Thus, even unexpected or unknown changes in the detected brain current patterns can be included and transmitted to another module, for example for further manual evaluation.

If the described sensor units are used in a vehicle, different reactions may be triggered depending on an evaluation of the measured magnetic fields. For example, the evaluation can check whether fatigue or a medical emergency is present. If a state is detected that is indicative of fatigue or even the onset of falling asleep, a warning may be issued. For example, a warning message can be shown on a display to prompt the driver to take a break. Additionally or alternatively, audible or visual warning signals may be provided, which may also prevent falling asleep.

Another application possibility for driver condition monitoring is the ability to respond quickly to emergencies. To this end, the brain magnetic field measurements may be checked for patterns indicative of certain diseases or critical health states. Depending on the urgency, an alert can then be issued again or emergency responses can be triggered. If a critical condition is detected, an emergency call signal could be transmitted via communication interfaces present in the vehicle, with could be used to alert which rescue services or other defined locations.

It is also possible to make certain driving functions dependent on the detected brain current patterns or the associated driver states. For example, it is conceivable to activate an automatic absolute speed limit if a driver is tired, or to activate a driver assistance system such as a lane keeping assist system or speed assist system. Optionally, this activation can only be proposed to the user and the actual activation can only be carried out after confirmation by the user.

In vehicles that are at least partially equipped with autonomous driving functions, certain driving functions may be made dependent on whether an awake and vital driver state is detected. For example, certain functions such as fully autonomous driving may only be enabled for a driver in an inconspicuous state.

If a critical state has been detected by the brain-magnetic field measurements and a driver no longer appears to be able to safely drive the vehicle, an autonomous driving function can, for example, initiate a departure to a parking lot or another safe position.

Similar reactions are also conceivable in other areas of application. Even when the sensor unit is integrated in a patient bed, warning signals can be output, in particular if a critical or unclear state is detected. The signals can be output in the immediate vicinity of the sensor unit or can also be transmitted to a monitoring unit of a clinic via communication means, for example. More detailed evaluations can also be made, which can then be displayed to medical personnel for a quick overview, e.g., an automatic or semi-automatic diagnostic classification of the measured brain signals.

In all variants, the detected sensor data may be evaluated, stored locally, and/or transmitted to further modules and systems. This makes it possible, for example, to transmit the associated measurement data from the magnetic field sensors directly to helpers on site, to a central monitoring center (e.g., in a clinic) or to an emergency call control center in the event of a critical medical state, so that this data can be used for further diagnostics. For example, a short-range radio interface, such as a Bluetooth interface, could be provided, via which the sensor data is transmitted from a memory element upon request or automatically when a suitable readout device approaches. In addition to the sensor data, the detected user states or other parameters can additionally or alternatively also be saved or transmitted. The magnetic field sensor data may also be supplemented by further data from separate sensors and modules, such as camera images, measured temperatures, stored information about the user such as names and important health data or information about the trip to date. A complete further state monitoring system may also be available and used in combination with the magnetic field data to decide on reactions, e.g., by evaluating eye movements of the user on camera images.

While gradiometers consisting of two similar magnetometers were described in the exemplary embodiments, gradiometer units may of course also be formed from different magnetometers, e.g., from an NV center magnetometer and a vapor cell magnetometer. Once again, interferences can be minimized or eliminated. Even in configurations in which a gradiometer arrangement is not used, but the individual magnetometers are evaluated separately, several different types of magnetometers can be used in one sensor unit. It is also possible to design similar or different magnetometers within a sensor unit at different detection frequencies and thus optimize them for different brain signals.

It should be understood that the exemplary embodiments described herein are intended only to illustrate the inventive principle and that it is possible to measure and evaluate the magnetic fields generated by brain currents in many other situations and areas of application in the manner described. In particular, such a system is not only suitable for motorists or motorcyclists, but also for all operators of safety-relevant equipment, such as train operators, pilots or machine operators. Likewise, the various evaluation options can be transferred to all examples. Thus, a system for detecting neural abnormalities in the medical setting for non-invasive monitoring of patients, but also in the mentioned vehicle applications or other situations, may be of interest. The different exemplary embodiments and elements can also be combined as desired. Thus, various gradiometer configurations and types of magnetometers may be used in any suitable holders for arrangement on a head. The further methods for position correction via a position reference module are also combinable with all variants and application areas. In addition to the embodiments described herein for the magnetometers with irradiation of a microwave field, other variants are also conceivable in which an applied external magnetic field comprises an evaluable resonance signal in an optically detected resonance spectrum due to the Zeeman effect and suitable spin polarizations.

1. A sensor unit for detecting brain current-induced magnetic fields in an unshielded environment, comprising:
a plurality of gradiometer units configured for arrangement around a head of a user, wherein each gradiometer unit has two magnetometers which are arranged at a fixed distance from each other, and further wherein each magnetometer has a sensor medium and is configured to detect a magnetic field strength at a measurement location by reading a spin resonance in the sensor medium dependent on the magnetic field strength,
at least one excitation light source configured to radiate light into the sensor media of the magnetometers, and
at least one signal processing unit configured to determine a magnetic field gradient at a gradiometer unit as a difference of the output signals of the two magnetometers of the gradiometer unit and to detect a time course of the magnetic field gradient.

2. The sensor unit according to claim 1, wherein at least one of the magnetometers comprises a nitrogen vacancy center magnetometer, wherein the sensor medium comprises a diamond crystal or a portion of a diamond crystal with nitrogen vacancy centers, and wherein the sensor unit further comprises at least one microwave source configured to generate a resonant field in the sensor medium and at least one photodetector configured to detect resonance-dependent sensor light from the sensor medium.

3. The sensor unit according to claim 2, wherein at least one of the gradiometer units is formed from two nitrogen vacancy center magnetometers, and wherein the same excitation light source and the same microwave source are associated with the two magnetometers of the at least one gradiometer unit.

4. The sensor unit according to claim 1, wherein at least one of the magnetometers comprises a vapor cell magnetometer, and wherein the sensor medium comprises an atomic spin-polarizable vapor in a cell, wherein the atomic vapor comprises one of the following: an alkali metal, a noble gas, and an alkali-metal azide.

5. The sensor unit according to claim 1, wherein the sensor unit comprises at least one holding device in which multiple gradiometers units are mounted such that they are arranged in operation at different locations near the head surface of a user.

6. The sensor unit according to claim 1, wherein the distance between the two magnetometers of a gradiometer unit is between 2 and 30 millimeters.

7. The sensor unit according to claim 1, wherein the holding device comprises one of the following: a head rest, a headgear, a headband, a cushion, and a head part of a recliner.

8. The sensor unit according to claim 1, wherein the head of the user can be received in the holding device such that the head remains freely movable relative to the holding device.

9. The sensor unit according to claim 1, wherein the sensor unit further comprises a position reference unit wearable on the head of a user and configured to determine a relative position or relative movement between the head of a user and the plurality of gradiometer units.

10. The sensor unit according to claim 9, wherein the position reference unit comprises at least one gyroscope or at least one element configured to generate a reference magnetic field that can be detected by magnetometers of the sensor unit.

11. A method for detecting brain current-induced magnetic fields using a sensor unit according to claim 1, the method comprising:
detecting magnetic field strengths through the magnetometers of the sensor unit in the head region of a user;
determining, for each gradiometer unit, a magnetic field gradient by forming a respective difference signal from the output signals of the associated magnetometers;
detecting a time course of the magnetic field gradients for each gradiometer unit; and
checking the time course of one or more magnetic field gradients for the occurrence of predetermined patterns.

12. The method according to claim 11, further comprising:
detecting a position reference signal for determining a relative position between the gradiometer units and the head of a user, and processing the position reference signal and the magnetic field gradients to determine a position-corrected path of the magnetic field gradients.

13. The method according to claim 12, wherein the signal for forming a position reference comprises one of the following: an additional brain current-induced magnetic field signal independent of a user's mental state; a gyroscope signal from one or more gyroscopes attached to the head of a user; and a magnetic field signal generated by a position reference unit attached to the head of a user.

14. The method according to claim 11, further comprising:
detecting, based on checking the time course, an undesirable state of the user, and
initiating responses based on the detection.

15. The method according to claim 11, wherein the undesirable state comprises one of the following: fatigue, stress, falling asleep, a neural disease, stroke, and epilepsy.

16. A computing unit, which is configured to carry out all method steps of a method according to claim 11.

17. A computer program which prompts a computing unit to carry out all method steps of a method according to claim 11 when it is executed on the computing unit.

18. A machine-readable storage medium with a computer program stored thereon according to claim 17.

19. The sensor unit according to claim 1, wherein the distance between the two magnetometers of a gradiometer unit is between 5 and 20 millimeters.
